(19) United States
(12) Patent Application Publication
(10) Pub. No.: US 2013/0197245 A1
(43) Pub. Date: Aug. 1, 2013

(54) Organometallic Molybdenum Acetylide Dioxo Complex And Process For The Preparation Thereof

(71) Applicants: Shubhangi Bhalchandra Umbarkar, Pune (Maharashtra) (IN); Mohan Keraba Dongare, Pune (Maharashtra) (IN); Ankush Biradar, Pune (Maharastra) (IN); Vaibhav Ravindrakumar Acham, Pune (Maharastra) (IN)

(72) Inventors: Shubhangi Bhalchandra Umbarkar, Pune (Maharashtra) (IN); Mohan Keraba Dongare, Pune (Maharashtra) (IN); Ankush Biradar, Pune (Maharastra) (IN); Vaibhav Ravindrakumar Acham, Pune (Maharastra) (IN)

(21) Appl. No.: 13/827,809

(22) Filed: Mar. 14, 2013

Related U.S. Application Data
(63) Continuation of application No. PCT/IN2011/000627, filed on Sep. 14, 2011.

(30) Foreign Application Priority Data
Sep. 14, 2010 (IN) 2177DEL2010

(51) Int. Cl. B01J 31/22; C07C 29/48; C07C 315/02; C07C 45/29; C07C 51/285; C07C 201/00; C07D 301/12; C07C 45/28

(52) U.S. Cl. CPC B01J 31/2295; C07D 301/12; C07C 29/48; C07C 45/28; C07C 45/294; C07C 51/285; C07C 201/00; C07C 315/02
USPC 549/525; 556/58; 568/833; 568/832; 568/363; 568/430; 562/418; 568/949; 568/28; 568/311

(57) ABSTRACT

An organometallic molybdenum acetylide dioxo complex of formula (η⁵-C₅H₅)MoO₂(—Cs≡CPh) and provides a simple, short, efficient process for the synthesis of organometallic molybdenum dioxo complex which is used as catalyst for a number of oxidation reactions.

### FIELD OF THE INVENTION

Present invention relates to organometallic molybdenum acetylide dioxo complex of formula (η⁵-C₅H₅)MoO₂(—C≡CPh). More particularly, the present invention relates to process using molybdenum oxide precursor for the preparation of organometallic molybdenum acetylide dioxo complex of formula (η⁵-C₅H₅)MoO₂(—C≡CPh). Present invention further relates to a process for the synthesis of the complex useful as a catalyst for various oxidation reactions.

### BACKGROUND OF THE INVENTION

Conventionally cyclopentadienyl transition metal dioxo organometallic complexes are prepared using corresponding transition metal carbonyl complexes involving multistep synthesis process as well as tedious work up procedure. These transition metal complexes are converted to corresponding dioxo/oxo-peroxo by treating with various oxidants like hydrogen peroxide, t-butyl hydrogen peroxide or cumyl hydrogen peroxide. Pentamethyl cyclopentadienyl tungsten acetylide dioxo and oxo-peroxo complexes have been prepared by pentamethyl_cyclopentadienyl tungsten tricorbonyl acetylide complex by treating with sulfuric acid and Hydrogen peroxide in toluene at room temperature for 24 hr. This process for the preparation of cyclopentadienyl transition metal dioxo organometallic complexes is cumbersome and results in the release of carbon monoxide as a by-product making it environmentally unacceptable. Further, the process is not viable for commercial applications because of use of costly metal carbonyls as starting material.

References may be made to thesis titled “Synthesis and characterization of heterogeneous rhenium and molybdenum catalysts. Applications in olefin metathesis and olefin epoxidation” by Draganco Veljanovski discloses various synthetic procedures for making of Cp*MoO₂Cl as shown in below scheme 1.

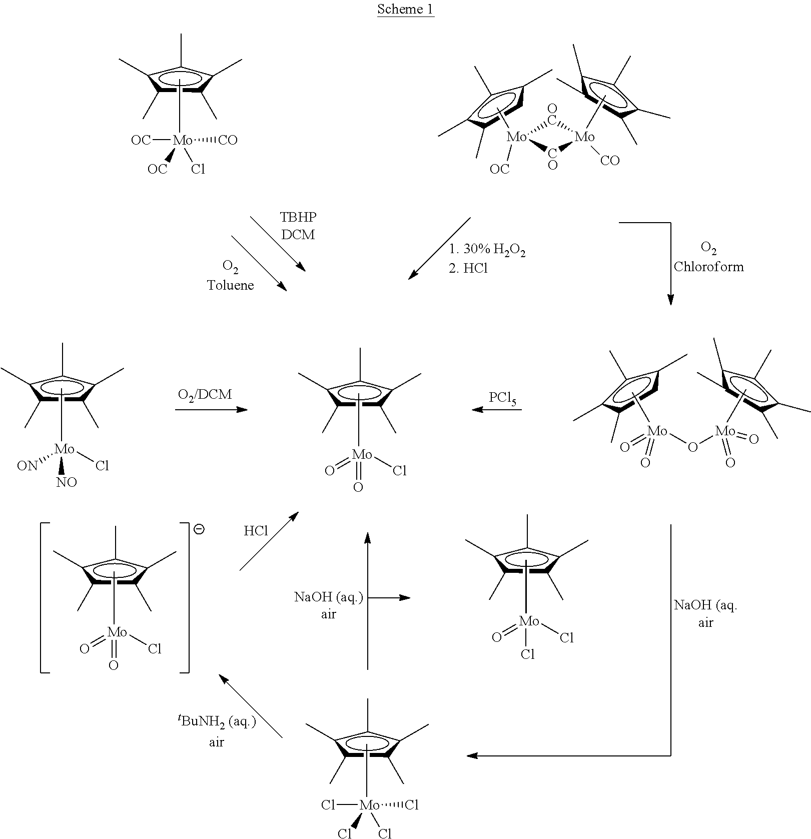

References may be made to an article titled “Selective cis-dihydroxylation of olefins using recyclable homogeneous molybdenum acetylide catalyst” by Ankush V. Biradar, Bhaskar R. Sathe et. al in Journal of Molecular Catalysis A: Chemical 285 (2008) 111-119 discloses preparation of CpMo(CO)₃(C≡CPh) wherein CpMo(CO)₃Cl and (H—C≡CPh) are reacted in presence of catalytic amount of CuI (5 mg) and diethyl amine (50 mL) as solvent. The catalyst is used for selective cis dihydroxylation of olefins with 95% conversion and selectivity of 86% using H₂O₂ as an oxidant and t-butanol as a solvent.

References may be made to article entitled “Selective oxidation of aromatic primary alcohols to aldehydes using molybdenum acetylide oxo-peroxo complex as catalyst” by Ankush V. Biradar et al in Tetrahedron Letters 50 (2009) 2885-2888 discloses preparation of CpMo(CO)₃(C≡CPh) wherein CpMo(CO)₃Cl and (H—C≡CPh) are reacted in presence of catalytic amount of CuI (5 mg) and diethyl amine (50 mL) as solvent. The catalyst is used for selective oxidation of aromatic primary alcohols to aldehydes.

But these are elaborate methods of greater than five steps, forms complex with one more oxygen molecule and use expensive starting material. The synthetic methods also suffer from disadvantages like decomposition during reaction and work-up which leads to low overall yields of the desired complex. Formation of Mo(VI) oxo/dioxo complex from corresponding Mo carbonyl complexes is always accompanied by liberation of environmentally hazardous CO gas due to dissociation of CO ligands. Further, the Mo complex products of these processes have the disadvantage of not being versatile in catalyzing a range of oxidation reactions.

Therefore, there is a need in the art to provide an efficient, economical process which results in improved yields and is also environment friendly to prepare organometallic molybdenum acetylide dioxo complex, (η⁵-C₅H₅)MoO₂(—C≡CPh).

It would be an advantage if the Mo complexes find use in various oxidation reactions, overcoming the restricted oxidation capacities of known Mo catalysts. [Ref.: Marta Abrantes, Ana M. Santos, Janos Mink, Fritz E. Kuhn, and Carlos C. Romao, Organometallics 2003, 22, 2112-2118; Anabela A. Valente, Jose Moreira, Andre D. Lopes, Martyn Pillinger, Carla D. Nunes, Carlos C. Romao, Fritz E. Kuhn and Isabel S. Gonc,alves, New. J. Chem., 2004, 28, 308-313]

### SUMMARY OF THE INVENTION

Main objective of the present invention is to provide organometallic molybdenum acetylide dioxo complex of formula (η⁵-C₅H₅)MoO₂(—C≡CPh).

Another objective of the present invention is to provide process using molybdenum oxide precursor for the preparation of organometallic molybdenum acetylide dioxo complex of formula (η⁵-C₅H₅)MoO₂(—C≡CPh) which is useful as a catalyst for various oxidation reactions.

Accordingly, present invention provides organometallic molybdenum acetylide dioxo complex of formula (η⁵-C₅H₅)MoO₂(—C≡CPh).

In an embodiment of the present invention, said organometallic molybdenum acetylide dioxo complex is useful as a catalyst for the oxidation of olefins, alcohols, anilines, sulfides and alkanes.

In another embodiment of the present invention, said complex is recyclable.

In yet another embodiment of the present invention, process for preparation of organometallic molybdenum acetylide dioxo complex of formula (η⁵-C₅H₅)MoO₂(—C≡CPh) comprising the steps of:
i. treating molybdenum trioxide with aqueous halo acids HX wherein X═F, Cl, Br or I, in the molar ratio of the trioxide to HX ranging between 1:6 to 1:15, at temperature in the range of 40° C. to 90° C. for period in the range of 2 to 5 hr to obtain aqua complex of dihalo dioxo molybdenum of formula MoO₂X₂.2H₂O wherein X═F, Cl, Br or I;
ii. adding dimethylsulphoxide or N,N-dimethylformamide to dihalo dioxo molybdenum as obtained in step (i) in the molar ratio ranging between 1:2 to 1:20 to form greenish adduct of formula MoO₂X₂.2DMSO or MoO₂X₂.2DMF respectively wherein X═F, Cl, Br or I;
iii. treating greenish adduct as obtained in step (ii) with sodium cyclopentadiene in molar ratio 1:1 to 1:20 followed by stirring at the rate of 100 to 1000 rpm to form cyclopentadiene dioxomolybdenum halo complex of formula CpMoO₂X wherein X═F, Cl, Br or I;
iv. treating cyclopentadiene dioxomolybdenum halo complex as obtained in step (iii) with phenyl acetylene to obtain molybdenum acetylide dioxo complex of formula (η⁵-C₅H₅)MoO₂(—C≡CPh).

In yet another embodiment of the present invention, catalytically active species (η⁵-C₅H₅) MoO(—C≡CPh) of the said organometallic molybdenum dioxo complex (η⁵-C₅H₅) MoO₂(—C≡CPh) formed after reacting with hydrogen peroxide is water soluble.

In yet another embodiment of the present invention, yield of molybdenum acetylide dioxo complex of formula (η⁵-C₅H₅)MoO₂(—C≡CPh) is in the range of 40 to 85%.

### BRIEF DESCRIPTION OF THE DRAWINGS

FIG. 1 is IR spectrum of the (η⁵-C₅H₅)MoO₂(—C≡CPh) catalyst.

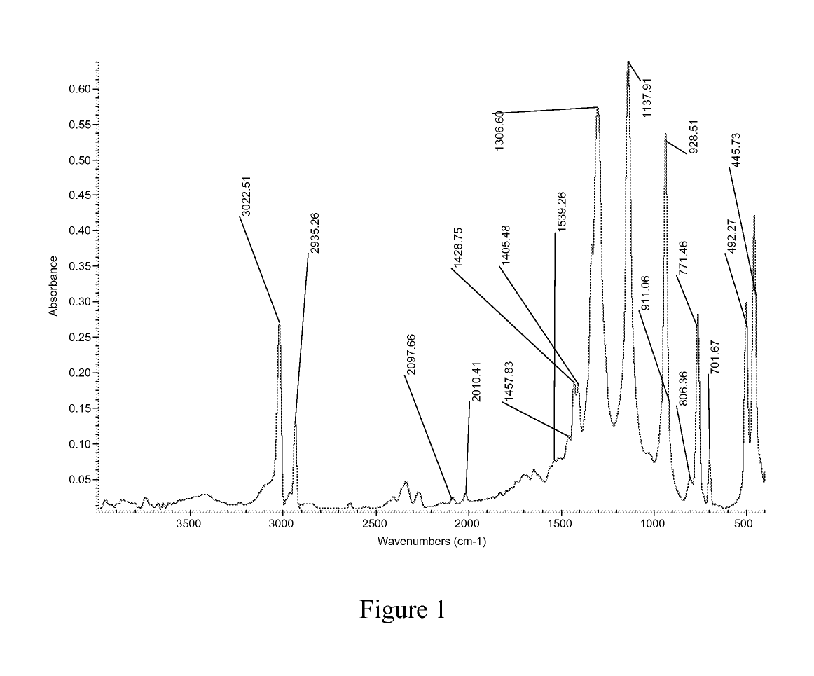

### DETAILED DESCRIPTION OF THE INVENTION

The present invention provides a simple process for the preparation of organometallic molybdenum dioxo complex (η⁵-C₅H₅)MoO₂(—C≡CPh) as a catalyst starting from simple precursor like molybdenum trioxide, MoO₃ instead of molybdenum hexacarbonyl as disclosed in prior art. The process comprises:
a. Preparing aqua complex of dihalo dioxo molybdenum (MoO₂X₂.2H₂O) treating molybdenum trioxide with an aqueous acid, H—X wherein X═F, Cl, Br, I;
b. Adding dimethylsulphoxide or N, N Dimethyl formamide to dihalo dioxo molybdenum of step a to form greenish adduct, MoO₂X₂.2DMSO or MoO₂X₂.2DMF respectively;
c. Treating MoO₂X₂.2DMSO or MoO₂X₂.2DMF with freshly prepared sodium cyclopentadiene and stirring to form cyclopentadiene dioxo molybdenum halo complex (CpMoO₂X); and
d. Treating CpMoO₂X with phenyl acetylene to form (η⁵-C₅H₅)MoO₂(—C≡CPh).

The product formed was confirmed by FTIR spectroscopy using Nicolet 870 Nexus FTIR spectrometer.

### IR Data for (η⁵-C₅H₅)MoO₂(—C≡CPh)

3022 (C—H stretch of Ph ring), 2935 (C—H stretch of Cp ring), 2097 cm⁻¹ (C≡C), 1725, 1707 (Mo—C stretch of Cp ring), 1539, 1457, 1428, 1407 (C═C stretch of Cp and Ph ring), 928 (cis-MoO₂ symmetric stretch), 911 (cis-MoO₂ antisymmetric stretch)

Such organometallic molybdenum dioxo complex finds use as catalysts for various reactions.

The catalyst is active for oxidation reactions such as oxidation of cyclohexene to cis diol similar to Sharpless catalyst. The catalyst is active for oxidation reactions such as oxidation of alcohols, anilines, olefins, sulfides and such like.

The catalyst has shown activity for oxidation reactions and even though it is homogeneous catalyst, being water soluble it can be recovered after the oxidation reaction by dissolving in water and separating it from organic phase for recovery and recycle, as exemplified herein with reference to example 12. The complex of the invention becomes water soluble on dissolution after reacting with hydrogen peroxide and is recovered from the oxidation reactions from the aqueous layer.

### EXAMPLES

The following examples are given by way of illustration only and therefore should not be constrained to limit the scope of the present invention.

### Example 1

### Synthesis of Dioxo Molybdenum Cyclopentadienyl Acetylide Complex CpMoO₂(C≡CPh)

Method 1

Molybdenum trioxide (MoO₃, 1.44 g, 10 mmol) was treated with conc. aqueous hydrochloric acid (7 ml, 35%) at 60° C. for 2 h to form aqua complex of dichloro dioxo molybdenum (MoO₂Cl₂.2H₂O). In the same reaction mixture was added 2.5 ml of dimethylsulphoxide (DMSO) to form the greenish adduct MoO₂Cl₂.2DMSO. MoO₂Cl₂.2DMSO was treated with sodium cyclopentadiene (CpNa, synthesized by reaction of sodium (253 mg, 11 mmol) with freshly cracked cyclopentadiene (743 mg, 11 mmol) in THF) and stirred for 3 h to form cyclopentadiene dioxomolybdenum chloride complex (CpMoO₂Cl). Another round bottom flask was charged with phenyl acetylene ((PhC≡CH, 1.10 g, 11 mmol) using copper (I) iodide (CuI, 5 mg) and diethyl amine (40 ml) and stirred for 30 min. This phenyl acetylene mixture was added to the first flask and stirred for 3 h at 30° C. to form CpMoO₂(—C≡CPh) (2.03 g), yield=69.05%.

### Example 2

### Synthesis of Dioxo Molybdenum Cyclopentadienyl Acetylide Complex CpMoO₂(C≡CPh)

Method 2

Molybdenum trioxide (MoO₃, 1.44 gm, 10 mmol) was reacted with conc. aqueous hydrochloric (7 ml, 35%) acid at 60° C. for 2 h to form aqua complex of dichloro dioxo molybdenum (MoO₂Cl₂.2H₂O). In the same reaction mixture was added 2.5 ml of N,N-dimethyl formamide (DMF) to form the greenish adduct MoO₂Cl₂.2DMF. MoO₂Cl₂.2DMF was treated with sodium cyclopentadiene (CpNa, synthesized by reaction of sodium (253 mg, 11 mmol) with freshly cracked cyclopentadiene (743 mg, 11 mmol) in THF) and stirred for 3 h to form cyclopentadiene dioxomolybdenum chloride complex (CpMoO₂Cl). To this reaction mixture was added preformed sodium phenyl acetylide (prepared by addition of sodium (253 mg, 11 mmol) to phenyl acetylene (1.10 g, 11 mmol) in THF) solution at −20° C. to form CpMoO₂(—C≡CPh) (1.9 g)), yield=64.62%.

### Example 3

### Synthesis of Dioxo Molybdenum Cyclopentadienyl Acetylide Complex CpMoO₂(C≡CPh)

Method 3

Molybdenum trioxide (MoO₃, 1.44 gm, 10 mmol) was reacted with conc. aqueous hydrochloric (7 ml, 35%) acid at 60° C. for 2 h to form aqua complex of dichloro dioxo molybdenum (MoO₂Cl₂.2H₂O). This complex was extracted with diethyl ether (30 ml×5). The combined ether layer was concentrated under reduced pressure. To the same solution was added 50 ml dried THF and remaining ether was removed under reduced pressure. The same solution was added to preformed sodium cyclopentadiene solution (CpNa, synthesized by reaction of sodium (253 mg, 11 mmol) with freshly cracked cyclopentadiene (743 mg, 11 mmol) in THF) at −78° C. and was stirred for 3 h to form cyclopentadiene dioxomolybdenum chloride complex (CpMoO₂Cl). Another round bottom flask was charged with phenyl acetylene ((PhC≡CH, 1.10 g, 11 mmol) using copper (I) iodide (CuI, 5 mg) and diethyl amine (40 ml) and stirred for 30 min. This phenyl acetylene mixture was added to the first flask and stirred for 3 h at 30° C. to form CpMoO₂(—C≡CPh) (2.03 g)), yield=69.04%

### Example 4

### Synthesis of Dioxo Molybdenum Cyclopentadienyl Acetylide Complex CpMoO₂(C≡CPh)

Method 4

Molybdenum trioxide (MoO₃, 1.44 gm, 10 mmol) was reacted with conc. aqueous hydrochloric (7 ml, 35%) acid at 60° C. for 2 h to form aqua complex of dichloro dioxo molybdenum (MoO₂Cl₂.2H₂O). This complex was extracted with diethyl ether (30 ml×5). The combined ether layer was concentrated under reduced pressure. To the same solution was added 50 ml dried THF and remaining ether was removed under reduced pressure. The same solution was added to preformed sodium cyclopentadiene solution (CpNa, synthesized by reaction of sodium (253 mg, 11 mmol) with freshly cracked cyclopentadiene (743 mg, 11 mmol) in THF) at −78° C. and was stirred for 3 h to form cyclopentadiene dioxomolybdenum chloride complex (CpMoO₂Cl). To this reaction mixture was added preformed sodium phenyl acetylide (prepared by addition of sodium (253 mg, 11 mmol) to phenyl acetylene (1.10 g, 11 mmol) in THF) solution at −20° C. to form CpMoO₂(—C≡CPh) (1.9 g)), yield=64.62%.

### Example 5

### Synthesis of Dioxo Molybdenum Cyclopentadienyl Acetylide Complex CpMoO₂(C≡CPh)

Method 5

Sodium molybdate (Na₂MoO₄.2H₂O; 2.12 g, 8.8 mmol) was reacted with concentrated aqueous HBr (15 ml) at 30° C. till dissolution of Na₂MoO₄. The solution was extracted with three 50 ml portions of diethyl ether. The combined extracts were concentrated under vacuum to approximately 15 ml and an ether solution of dimethyl formamide (1.50 g, 20.5 mmol) was added drop wise. A yellow precipitate was obtained and the mixture was stirred for 30 min before the precipitate was filtered, washed with ether and dried over P₂O₅. MoO₂Br₂ (OCHNMe₂)₂. Yellow solid. Yield: (3.46 g, 91%); m.p.: 54° C. MoO₂Br₂.2DMF was treated with sodium cyclopentadiene (CpNa, synthesized by reaction of sodium (253 mg, 11 mmol) with freshly cracked cyclopentadiene (743 mg, 11 mmol) in THF) and stirred for 3 h to form cyclopentadiene dioxomolybdenum chloride complex (CpMoO₂Cl). Another round bottom flask was charged with phenyl acetylene ((PhCECH, 1.10 g, 11 mmol) using copper (I) iodide (CuI, 5 mg) and diethyl amine (40 ml) and stirred for 30 min. This phenyl acetylene mixture was added to the first flask and stirred for 3 h at 30° C. to form CpMoO₂(—C≡CPh) (2.03 g)), yield=78.46%.

### Example 6

### Synthesis of Dioxo Molybdenum Cyclopentadienyl Acetylide Complex CpMoO₂(C≡CPh)

Method 6

Molybdenum trioxide (MoO₃, 1.30 gm, 9 mmol) was reacted with conc. aqueous hydrochloric (7 ml, 35%) acid at 60° C. for 2 h to form aqua complex of dichloro dioxo molybdenum (MoO₂Cl₂.2H₂O). In the same reaction mixture was added 2.5 ml N,N-dimethyl formamide (DMF) to form the greenish adduct MoO₂Cl₂.2DMF. To a THF solution of MoO₂Cl₂.2DMF was added freshly prepared THF solution of CpNa (CpNa, synthesized by reaction of sodium (210 mg, 9 mmol) with freshly cracked cyclopentadiene (594 mg, 9 mmol) in THF) at 30° C. and the reaction mixture was stirred for 3 h at 30° C. In second flask lithium salt of phenyl acetylene was prepared by mixing n-butyl lithium (7.2 ml 2.5 molar solution in hexane, 18 mmol) and phenyl acetylene (0.924 g, 9 mmol) at −78° C. for 1 h. The solution of CopMoO₂Cl prepared in first step was added to THF solution of freshly prepared lithium salt of phenyl acetylene at −78° C. and the solution was warmed up to 30° C. and further stirred for 3 h at 30° C. The unreacted butyl lithium was quenched by NH₄OH solution and the solvent was removed under vacuum. The reaction mixture was extracted in dichloromethane and the solvent was removed in vacuum to obtain CpMoO₂(—C≡CPh) (1.25 g)), yield=47.24%.

### Example 7

### Oxidation of Olefin (Cyclohexene)

The cyclopentadienyl dioxo molybdenum acetylide complex as prepared in example 1 was used as catalyst for catalytic oxidation of cyclohexene. Cyclohexene (0.820 g, 10 mmol), hydrogen peroxide (11 mmol, 30% aqueous) and catalyst (0.1 mmol, 30 mg) was heated to 80° C. in acetonitrile for 8 h giving 100% conversion of cyclohexene. The products obtained were cyclohexene oxide (40%), trans cyclohexane diol (17%), 2-cyclohexenol (23%), 2-cyclohexenone (20%).

### Example 8

### Oxidation of Alcohols

The cyclopentadienyl dioxo molybdenum acetylide complex as prepared in example 2 was used as catalyst for catalytic oxidation of activated alcohols. Benzyl alcohol (10 mmol), hydrogen peroxide (20 mmol) and catalyst (0.1 mmol, 30 mg) was heated at 80° C. for 8 h giving 86% conversion with 92% selectivity for benzaldehyde and 8% selectivity for benzoic acid.

### Example 9

### Oxidation of Anilines

The cyclopentadienyl dioxo molybdenum acetylide complex as prepared in example 3 was used as catalyst for catalytic oxidation of various anilines. Aniline (10 mmol), hydrogen peroxide (20 mmol) catalyst (0.1 mmol, 30 mg) was stirred at 30° C. for 12 h giving 97% aniline conversion and 97% selectivity for nitrosobenzene.

### Example 10

### Oxidation of Sulfides

The cyclopentadienyl dioxo molybdenum acetylide complex as prepared in example 4 was used as catalyst for catalytic oxidation of various sulfides including refractory sulfides using hydrogen peroxide. 4,6-Dimethyldibenzothiophene (4,6-DMDBT, 10 mmol), hydrogen peroxide (20 mmol) and catalyst (0.1 mmol, 30 mg) was heated at 100° C. for 8 h giving 75% conversion with 100% selectivity for corresponding sulphone.

### Example 11

### Oxidation of Alkyl Benzene

The cyclopentadienyl dioxo molybdenum acetylide complex as prepared in example 1 was used as catalyst for catalytic oxidation of ethyl benzene. Ethyl benzene (10 mmol), t-butyl hydrogen peroxide (TBHP, 20 mmol) and catalyst (0.1 mmol, 30 mg) were heated at 80° C. in acetonitrile (10 g) for 20 h giving 81% conversion and 85% selectivity for acetophenone.

### Example 12

### Catalyst Recycles Study

After completion of the reaction in example 9, the solvent was removed under reduced pressure. Ethyl acetate (20 ml) was added to the reaction mixture. Aqueous layer containing catalysts and organic layer containing product and unreacted reactant was separated using separating funnel. The aqueous phase containing catalyst was concentrated to 2 ml. To this 2 ml aqueous solution containing catalyst, fresh charge of ethyl benzene (10 mmol), t-butyl hydrogen peroxide (TBHP, 20 mmol) and acetonitrile (10 g) was added and was heated at 80° C. for 20 h. The catalyst was recycled five times using the above procedure. The ethyl benzene conversion and product selectivity for five recycle are given below.

| Recycle | Conversion | Selectivity (%) | | Acetophenone |
|---|---|---|---|---|
| no. | (%) | Acetophenone | 1-phenylethanol | yield, % |
| 0 | 80 | 85 | 15 | 68.0 |
| 1 | 79.5 | 84.7 | 15.3 | 67.3 |
| 2 | 79.2 | 84.4 | 15.6 | 66.8 |
| 3 | 78.8 | 84.0 | 16.0 | 66.2 |
| 4 | 78.4 | 84.0 | 16.0 | 65.5 |
| 5 | 78.0 | 84.0 | 16.0 | 65.5 |

### ADVANTAGES OF THE INVENTION

1. Simple and short process.
2. Improved yield of process.
3. Starting material is cheap.
4. Versatile to catalyze range of oxidation reactions.
5. Recyclable catalyst.

What is claimed is:

1. Organometallic molybdenum acetylide dioxo complex of formula (η⁵-C₅H₅)MoO₂(—C≡CPh).

2. Organometallic molybdenum acetylide dioxo complex as claimed in claim 1 is useful as catalyst for the oxidation of olefins, alcohols, anilines, sulfides and alkanes.

3. Organometallic molybdenum acetylide dioxo complex as claimed in claim 1, wherein said complex is recyclable.

4. Organometallic molybdenum acetylide dioxo complex as claimed in claim 1, wherein catalytically active species (η⁵-C₅H₅) MoO(O₂)(—C≡CPh) of the said organometallic molybdenum dioxo complex (η⁵-C₅H₅) MoO₂(—C≡CPh) formed after reacting with hydrogen peroxide is water soluble.

5. A process for preparation of organometallic molybdenum acetylide dioxo complex of formula (η⁵-C₅H₅)MoO₂(—C≡CPh) as claimed in claim 1 and the said process comprising the steps of:
i. treating molybdenum trioxide with aqueous halo acids HX wherein X═F, Cl, Br or I in the molar ratio of the trioxide to HX ranging between 1:6 to 1:15 at temperature in the range of 40° C. to 90° C. for period in the range of 2 to 5 hr to obtain aqua complex of dihalo dioxo molybdenum of formula MoO₂X₂.2H₂O wherein X═F, Cl, Br or I;
ii. adding dimethylsulphoxide or N,N-dimethylformamide to dihalo dioxo molybdenum as obtained in step (i) in the molar ratio ranging between 1:2 to 1:20 to form greenish adduct of formula MoO₂X₂.2DMSO or MoO₂X₂.2DMF wherein X═F, Cl, Br or I;
iii. treating greenish adduct as obtained in step (ii) with sodium cyclopentadiene in molar ration of 1:1 to 1:20 followed by stirring at the rate of 100 to 1000 rpm to form cyclopentadiene dioxomolybdenum halo complex of formula CpMoO₂X wherein X═F, Cl, Br or I;
iv. treating cyclopentadiene dioxomolybdenum halo complex as obtained in step (iii) with phenyl acetylene to obtain molybdenum acetylide dioxo complex of formula (η⁵-C₅H₅)MoO₂(—C≡CPh).

6. The process as claimed in claim 5, wherein yield of molybdenum acetylide dioxo complex of formula (η⁵-C₅H₅)MoO₂(—C≡CPh) is in the range of 40 to 85%.

7. A process for the oxidation of organic compounds using organometallic molybdenum acetylide dioxo complex of formula (η⁵-C₅H₅)MoO₂(—C≡CPh) as claimed in claim 1, wherein said process comprises heating the mixture of organic compound, hydrogen peroxide and the complex at temperature in the range of 75 to 85° C. for period in the range of 7 to 9 hrs to obtain oxidized product.

8. The process as claimed in claim 1, wherein the organic compound is selected from a group consisting of olefins, alcohols, anilines, sulfides and alkanes.
